(19) United States
(12) Patent Application Publication
(10) Pub. No.: US 2024/0294765 A1
(43) Pub. Date: Sep. 5, 2024

(54) Method of making a composite pigment based on red, purple, orange and brown dyes with an antioxidant effect and the resulting composite pigment

(71) Applicants: Gurgen SARDARYAN, Pardubice (CZ); Garegin SARDARYAN, Pardubice (CZ); GESMED BIOTEC S.R.O, Praha 1 (CZ)

(72) Inventors: Gurgen SARDARYAN, Pardubice (CZ); Garegin SARDARYAN, Pardubice (CZ)

(21) Appl. No.: 18/279,072

(22) PCT Filed: Dec. 8, 2021
(86) PCT No.: PCT/CZ2021/000055

§ 371 Date: Aug. 28, 2023

(51) Int. Cl. C09B 61/00

(52) U.S. Cl. CPC C09B 61/00

(57) ABSTRACT

The solution concerns a method of producing a composite pigment based on red, purple, orange and brown dyes with an antioxidant effect from a cultivation biomass of Penicillium oxalicum var. Armeniaca CCM 8242 and CCM 8374 microorganism strains, especially for the food, pharmaceutical and cosmetic industries, characterised by the initial biomass containing at least 10% w/w of a mixture of α and β glucans, 3% w/w niacin, 7% w/w pantothenic acid and 1.5% w/w pyridoxine in the dry matter.

The acquired composite pigment is characterised by typical maxima in the spectrophotometric diagram presented by molecular absorption spectrophotometry in the visible spectrum in wavelength range 400 nm to 800 nm, and that two maxima in a water solution, pH values of 7 to 7.5, where the first maximum on Lambda max₁ is A=494 nm and the second highest maximum on Lambda max₂ is A=420 nm, and two maxima in an alcohol solution, where the first maximum on Lambda max₁ is A=502 nm and the second highest maximum on Lambda max₂ is A=415 nm.

### TECHNICAL FIELD

The technical solution deals with the method of making a composite pigment based on red, purple, orange and brown dyes that has an antioxidant effect and is used especially in the food, pharmaceutical and cosmetic industries.

### PRIOR ART

Czech patent No. 285721 describes the microscopic fungus Penicillium oxalicum var. Armeniaca CCM 8242 acquired from the soil of the valley below Mount Ararat; patent No. 302696 refers to microscopic fungus CCM 8374. Both fungi produce an exogenous red pigment which offers various forms of use in the food, pharmaceutical and cosmetic industries.

The current uses of the above-mentioned fungal strains result in lower yield of pigment and its unstable quality, which is not desired.

Both above-mentioned strains were kept at the CCM International Depository Authority site, Czech Collection of Microorganisms at Masaryk University in Brno, Czech Republic (CR).

### Nature of the Invention

The shortcomings mentioned above have been removed by the method of preparation of a composite pigment based on red, purple, orange and brown dye with an antioxidant effect from a biomass cultivation of Penicillium oxalicum var. Armeniaca CCM 8242 and CCM 8374 strains of microorganisms where the gist of the solution lies in the initial biomass containing at least 10% w/w mixture of α and β glucans, 3% w/w niacin, 7% w/w pantothenic acid and 1.5% w/w pyridoxine in dry matter.

According to the invention, the composite pigment based on red, purple, orange and brown dyes with an antioxidant effect, prepared from a biomass of Penicillium oxalicum var. Armeniaca CCM 8242 and CCM 8374 strains microorganisms, especially for the food, pharmaceutical and cosmetic industries is also the subject matter of the invention based on presenting characteristic maximum values in the spectrophotometric diagram produced by molecular absorption spectrophotometry in the visible part of the spectrum at wavelength range of 400 nm to 800 nm-two maxima in a water solution, pH 7 to 7.5, where the first maximum on Lambda max₁ is A=494 nm and the second highest maximum on Lambda max₂ is A=420 nm, and two maxima in an alcohol solution, where the first maximum on Lambda max₁ is A=502 nm and the second highest maximum on Lambda max₂ is A=415 nm.

The biomass for the production of antioxidant composite pigments according to the invention with the application of Penicillium oxalicum var. Armeniaca CCM 8242 and CCM 8374 strains of microorganisms also contains glucans in a minimal amount from 10% w/w.

The new composite pigments are produced by the fermentation of an inoculum from which pigments with an antioxidant effect are isolated.

The composite pigments according to the invention are used especially in the food industry, e.g., in smoked meats and as a food supplement, and also in the pharmaceutical and cosmetic industries where they stabilise and colour the product. The antioxidant effect of these pigments prolongs considerably the use-by-date and the life of all the products to which they are added.

The composite pigments according to the invention and their production offer a final product with a reproducible stable composition. This could not be taken for granted so far. Their production now takes place under increased saturation of the fermentation medium with air, treatment of the fermentation medium with yeast extract (e.g., HY YEST 412 Kerry) and adjustment of the size of the pores of the microfiltration equipment. By modifying the fermentation process, we get a mycelium biomass containing substances that are beneficial for health as it contains at least 10% w/w glucan; see FIG. 2.

### EXPLANATION OF DRAWINGS

FIG. 1 presents a diagram of spectrophotometric measurement of the composition of the final basic product (mass spectrophotometry)—a red pigment. The following pigments—purple, orange and brown—are determined chromatographically.

FIG. 2 is a diagram showing the FTIR spectrum of fungal glucan and mycelia Penicillium oxalicum and Pleurotus (oyster mushroom), where the full line represents glucan and the ornamental line represents Penicillium oxalicum. The spectrum of the sample lies within wavenumbers 4000 to 450 cm⁻¹, resolution 2 cm⁻¹. This is a comparison of the fungal glucan spectrum with glucans contained in the Penicillium oxalicum var. Armeniaca biomass.

The diagram in FIG. 3 shows the spectrum produced by a mass spectrophotometer from an analysis of a sample acquired from experimental fermentation number 2 (natural red batch 2 (NR)) performed by the Microbial Institute of the CR Academy of Sciences, CR.

The diagram in FIG. 4 shows the spectrum from a mass spectrophotometer of an analysis of a sample acquired from an experimental pilot plant production from fermentation number 125 (natural red batch 125 (NR)) performed at the Food Research Institute Prague, CR.

The composite pigments according to the invention were tested in operation in the laboratories of the Microbial institute of the Academy of Sciences of CR with good results.

### EXAMPLES OF IMPLEMENTATION OF THE INVENTION

### Example 1

The production of composite pigments according to the invention is carried out from a biomass containing at least 10% w/w glucans after the fermentation of Penicillium oxalicum var. Armeniaca CCM 8242 and CCM 8374 microbial strains, following the basic production scheme:
RO preparation of DEMI water for the production
Steam generator and preparation of live steam for sterilisation
Air compressor for the production
Sanitizing equipment
Cooling system
Microbiological laboratory, work with the strain and preparation of the inoculum
Biotech fermenter—acquiring the active ingredient
Product processing line—centrifuge, MF, NF—isolation of the active ingredient
Spray dryer—drying the product on a suitable carrier

The entire fermentation process takes place under sterile conditions.

Production starts in the microbiological lab where the strain of the Penicillium oxalicum var. Armeniaca CCM 8242 and CCM 8374 microscopic fungus is activated from its lyophilized state. It is inoculated into petri dishes and then cultivated in a thermostat at a temperature of 28 to 29° C. for 5 days.

The mycelia assessment criteria are:
Uniform velvety surface of the mycelia
Green-colored mycelia
abundant sporulation
the color of the agar under the mycelia is dark red

We scrape the homogeneous matter of the spores with the mycelium with a laboratory loop from the surface of the petri dish and transfer it sterilely into a 500 ml Erlan Mayer flask. We cultivate the inoculum in flasks in a shaker rotating at 220 to 240 rotations per minute for 36 to 48 hours.

The fermentation medium for inoculation is a yeast extract (e.g., HY YEST 412 Kerry) in the amount of 6 g/l of medium, beet sugar—sucrose in the amount of 18 g/l of medium, and agar-agar mass 20 g/l of medium.

The acquired selected inoculum is transferred sterilely into an inoculation fermenter. The volume of the charge of the inoculation fermenter is approx. 0.1% to 2% of the volume of the process fermenter

The conditions of the process in the inoculation fermenter are as follows:

Temperature inside the unit 28 to 29° C.; pressure inside the unit 0.02 to 0.03 MPa; consumption of air 15 to 30 m³ of air/m³ of the fermentation medium per hour; stirring with a paddle-wheel stirrer at speed of 250 rot/min; pH of medium 5.8 to 6.2. The fermentation medium for inoculation is yeast extract HY YEST 412 Kerry in the amount of 6 g/l of medium, beet sugar—sucrose 18 g/I medium.

The activity of the fungus must be preserved in test tubes in a regular inoculation cycle every 3 months. The fungus must be kept at temperatures of 3 to 5° C.

The inoculum is cultivated for 36 to 48 hours; the mycelium must have a cottonwool-like structure with long fibres and abundant sporulation and be evenly suspended in the fermentation medium. During fermentation we monitor pH values, the amount of dye, amount of mycelia biomass and the microbial purity of the sample—monoculture, moulds. After the passage of the above-mentioned period, we transfer sterilely the inoculum into the sterilised fermentation medium in the process fermenter.

The conditions in the process fermenter are as follows:

The volume of the inoculum in the process fermenter is approx. 0.1% to 3% of the volume of the process fermenter.

The temperature inside the unit is 28 to 29° C., pressure inside the unit is 0.07 to 0.08 MPa, consumption of air 30 to 50 m³ of air per m³ of the fermentation medium per hour, stirring with a paddle-wheel stirrer at speed of 250 to 400 rotations per minute, pH of the medium 5.8 to 6, oxygen solubility 80 to 100%.

The fermentation medium for inoculation is yeast extract HY YEST 412 Kerry in the amount of 6 g/litre of medium, beet sugar—sucrose in the amount of 18 g/l of medium, PPG defoamer as needed.

The mycelium is thus cultivated for 68 to 72 hours, until the sucrose in the medium is used up; during fermentation we monitor pH, the amount of residual sucrose, the amount of pigment, amount of mycelia biomass and the microbial purity of the sample—monoculture, mould.

Once fermentation is finished, we abruptly increase the temperature in the fermenter up to 45° C. to 50° C., adjust the pH with ammonia water to pH 8.5 to 9.5 and stir at a speed of 35 to 40 rot/min.

Next come the finalising steps, i.e., removal of the mycelia using a centrifuge, speed 15 000 rot/min., or possibly pressure filtration from the fermentation liquid containing the required product—red pigments. The mycelia content is approx. 3 to 5% of the volume of the charge. This is followed by cleaning the medium by ridding it of fragments of mycelia and insoluble matter by running it through a microfiltration device (size of membrane pores 0.45 to 0.60 μm) and in the final stage concentrating the product to a volume of 1/10 of the volume of the charge in a nanofiltration unit (pore size 300 to 350 Daltons).

The concentrated product is then spray-dried on a suitable carrier. The temperature of the sprayed material is 35 to 45° C., pH value is 9.0 to 9.5. The air temperature at the inlet to the drying chamber is 200° C.; the temperature at the outlet from the drying chamber is 98° C.

The result is a slightly bitter-tasting red powder, together with purple, orange and brown pigments determined by chromatography.

The analysis of the final product—a red pigment after fermentation of Penicillium oxalicum var. Armeniaca CCM 8242 and CCM 8374 strains—produced in optimal fermentation conditions showed a yield of 6 to 10 g of red pigment per litre of medium. The absorption value calculated by the FAO-proposed method is 1.05. In this case the concentration of red pigment is determined according to the following formula:

$$\% = \frac{100 \times A \times 100}{1.05 \times W} ,$$

where
A lambda max. absorption at 494 nm,
W mass of the sample of crystalline pigment 100 mg,
1.05 absorption rate constant.

The acquired red pigment was analysed spectrometrically by. molecular absorption spectrophotometry in the visible field of the electromagnetic spectrum with a frequency range of 400 nm to 800 nm, where 2 characteristic maxima were achieved in a water solution, pH 7 to 7.5, and the first maximum on Lambda max₁ was A=494; the second highest maximum on Lambda max₂ was A=420 nm.

Another spectrometry was carried out on an alcohol solution of the red pigment, where the first maximum on Lambda max₁ was A=502 nm, and the second highest maximum on Lambda max₂ was A=415 nm.

The quality (purity) of the pigment is reflected in the A₁/A₂=1.3 to 1.5 value at which antioxidant activity is 2.65.

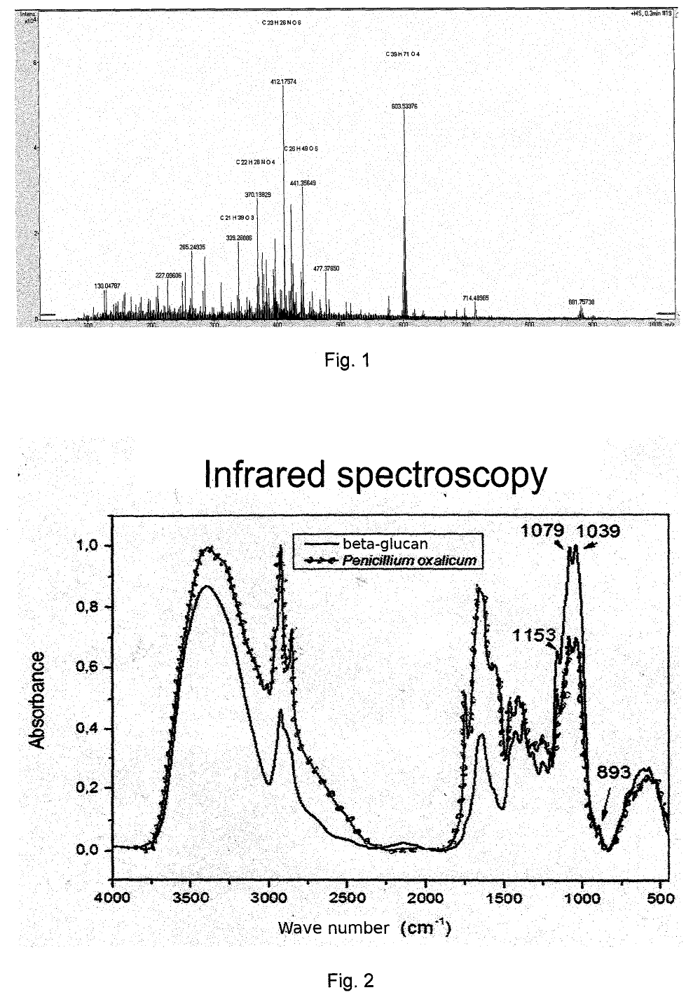

The quality of the slightly bitter-tasting red powder, simultaneously with purple, orange and brown pigments, is illustrated by the diagram in FIG. 1.

### Example 2

Production proceeded as in Example 1, but with the following modifications:

The source of nitrogenous substances used in the fermentation medium was yeast extract HY-YEST 412 Kerry. During aeration, 30 to 50 m³ of air per m³ of fermentation medium per hour was used in the fermentation process. The temperature was 28 to 29° C. and the medium was stirred at a speed of 250 to 400 rotations per minute. The fermented medium showed a red colouring as early as after 22 hours. Fermentation was completed in 68. to 72. hours. To finalise and process the product, microfiltration using membranes of pore size 45 to 0.60 μm and nanofiltration using membranes of pore size 300 to 350 Dalton was used.

### Example 3

The biomass used in the production of composite pigments according to the invention must contain at least 10% w/w glucans to achieve good utilisation in nutrition as required.

Table 1 shows the composition of the mycelia=biomass (can be used for complex drying):

TABLE 1

| content/factor | mycelium |
|---|---|
| Dry matter | g/100 g dry matter |
| Ash | 6.5 |
| Fat | 3.9 |
| Proteins | 32.1 |
| Total food fibre | 37.0 |
| Soluble food fibre | 26.8 |
| Insoluble food fibre | 10.3 |
| content/factor | Mycelium |
| Vitamins in the biomass | g/100 g dry matter |
| Niacin | 3.7 |
| Pantothenic acid | 7.10 |
| Pyridoxine | 1.81 |

### Note:

Fat—origin defoamer
Proteins—probably slightly affected by addition of ammonia
Antioxidant activity (mg AA/1 g dry matter) (DPPH)=2.65

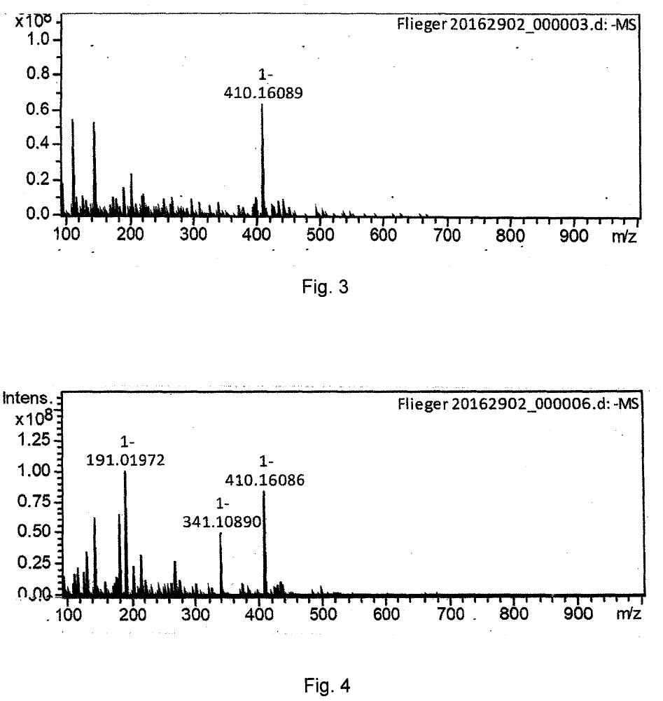

Table 2 compares the glucan content in biomass—see FIG. 2:

TABLE 2

| content/factor | Mycelium |
|---|---|
| Dry matter | g/100 g dry matter |
| Total glucans | 13.25 |
| α - glucans | 3.40 |
| β - glucans | 9.85 |

Fungi of the Pleurotus genus (lyophilized mycelia)

| | |
|---|---|
| Total glucans | 17% |
| α - glucans | 3% |
| β - glucans | 14% |

FIG. 2 shows the FT-IR spectra of fungal glucan and Penicillium oxalicum mycelia. The FT-IR spectrum of the sample was measured within a wavenumber range of 4000 to 450 cm⁻¹ with a 2 cm⁻¹ resolution and the spectra were compared with the spectrum of fungal glucan where the presence was confirmed of a glucan containing 1.3 and 1.6 of the bond; the sample also contains fats (region of 3000 cm¹) and proteins (region of 1700 cm⁻¹). The acquired composite pigments. according to the invention. in the form of a red pigment were subjected to analysis, as presented in FIG. 2.

Samples of composite pigments (Natural Red) marked as crude extract and original sample were tested using cellular-biological techniques (fluorescence microscopy, flow cytometry) on primary human skin fibroblasts. The experiment was performed twice for each sample; the acquired data did not show any significant scattering, this is a biological replicate.

### Method:

To achieve an optimal physiological state, the cells were defrosted from stock cultures kept in liquid nitrogen a week prior to the experiment. The cells were then 1× passaged to an optimal cellular density (confluence approx. 50%) in plates with 24 pits with an inserted covering glass (Nunc). To test bioactivity/toxicity, the reagents stored in a 50 mg/ml (4° C.) concentration were diluted under sterile conditions in a medium for tissue cultures (D-MEM, 10% FCS) and added in the required concentrations to the tested fibroblasts. The test ran for 24 h/72 h at 37° C., in a 5% CO₂ atmosphere.

For the flow cytometry (FACS), the cells were separated from the cultivation plate with the help of 0.1% trypsin solution, immediately marked with the DAPI fluorescent dye used to identify necrotic and apoptotic cells (HY YEST 412 Kerry) and subsequently analysed using FACS Aria (Becton Dickinson).

The data were analysed using FlowJo software.

For fluorescent microscopy, the cells were fixed with 4% PFA in PBS for 10 minutes, permeabilised with 0.01% Triton X-100 in PBS, RT, blocked with 5% BSA in PBS and marked with anti-Lamp-3 (MEM 259) monoclonal antibodies and fluorescent Alexa 594 phalloidin. The murine monoclonal antibody was then identified with the help of the secondary GAM-Alexa 488 antibody. Visualization was performed using an Olympus inverted fluorescence microscope and sensitive DP50 colour camera (40× lens).

### Flow Cytometry:

Dot-plot analysis shows the result of one of the replicates over 24-hour incubation with concentrations covering a span of 8 to 1000 micrograms/ml.

### Conclusion:

Flow cytometry did not detect any significant cytotoxicity in the concentration range of 8 to 1000 micrograms/mi. The highest tested concentration is extreme; only few secondary metabolite-type substances are comparably bio-neutral. The biological replicate produced similar data in both the 24-hour and 72-hour experiment.

### Fluorescence Microscopy:

Primary skin fibroblasts were tested for Natural Red extracts in the concentration range of 1 to 500 micrograms/ml. The integrity and pattern of the actin cytoskeleton (which is extremely sensitive to environmental effects) was visualized as well as the late endosomal/lysosomal vesicular system and nuclei.

### Conclusion:

The selected combination of characteristics is suitable for sensitive identification of disturbance of cellular physiology. The latter has not been recorded, maybe with the exception of the highest concentration which led to a slight change in adhesion and to cell elongation; the endosomal/lysosomal system was not affected and also no necrosis or apoptosis was observed (in either time aspect, i.e., 24 and 72 hours), in both fractions in the biological replicate.

The studied Natural Red fractions manifest extraordinary biocompatibility/absence of cytotoxicity to the primary cellular line of human skin fibroblasts. No significant cellular pathology was recorded within the broad concentration ranges applied over a number of days and tested by sensitive quantitative (flow cytometry) and also qualitative techniques (fluorescence microscopy).

### INDUSTRIAL APPLICABILITY

The solution concerns a new method of producing composite red, purple, orange and brown pigments with an antioxidant effect, resulting from the cultivation and fermentation of the biomass from Penicillium oxalicum var. Armeniaca CCM 8242 and CCM 8374 strains; further, it also concerns these new composite pigments for use in the food, pharmaceutical and cosmetic industries.

### List of Abbreviations Used in the Text:

FT-IR spectra—fluorescence infra-red spectrum detector
RO—reverse osmosis
DEMI—demineralised
MF—microfiltration
NF—nanofiltration
PPG—polypropylene glycol
DPPH—diphenyl picrylhydrazil
CO₂—carbon dioxide
FCS—fluorescence correlation spectroscopy
FACS—fluorescence flow cytometry
DAPI—4,6-diamidino-2-phenilindole—fluorescent dye
PFA—plasma proteins
PBS—buffer (phosphates) for tissue cultures
Triton X-100—non-ionogenic tenside
RT—reverse transcriptase
BSA—bovine serum albumin
MEM—monoclonal antibody
GAM-Alexa 488—name of antibody

1. Method of producing a composite pigment based on red, purple, orange and brown dyes with an antioxidant effect from a cultivation biomass of Penicillium oxalicum var. Armeniaca CCM 8242 and CCM 8374 microorganism strains, characterised in that, the initial biomass contains at least 10% w/w of a mixture of α and β glucans, 3% w/w niacin, 7% w/w pantothenic acid and 1.5% w/w pyridoxine in the dry matter.

2. Composite pigment based on red, purple, orange and brown dyes with an antioxidant effect, prepared from a biomass of Penicillium oxalicum var. Armeniaca CCM 8242 and CCM 8374 microorganism strains according to claim 1, especially for the food, pharmaceutical and cosmetic industries, characterised by typical maxima in the spectrophotometric diagram presented by molecular absorption spectrophotometry in the visible spectrum in the wavelength range of 400 nm to 800 nm, and that two maxima in a water solution with pH values of 7 to 7.5, where the first maximum on Lambda max₁ is A=494 nm and the second highest maximum on Lambda max₂ is A=420 nm, and two maxima in an alcohol solution, where the first maximum on Lambda max₁ is A=502 nm and the second highest maximum on Lambda max₂ is A=415 nm.
